(19) United States
(12) Patent Application Publication
(10) Pub. No.: US 2017/0232031 A1
(43) Pub. Date: Aug. 17, 2017

(54) Inhibitors of Zika Virus

(71) Applicant: Riboscience LLC, Palo Alto, CA (US)

(72) Inventors: Mark Smith, San Francisco, CA (US); Klaus G. Klumpp, West Chester, PA (US)

(21) Appl. No.: 15/434,191

(22) Filed: Feb. 16, 2017

Related U.S. Application Data
(60) Provisional application No. 62/296,024, filed on Feb. 16, 2016.

(51) Int. Cl. A61K 31/7072; A61K 45/06

(52) U.S. Cl. CPC A61K 31/7072; A61K 45/06

(57) ABSTRACT

The application provides methods for treatment or prophylaxis of Zika Virus mediated diseases with compounds of Formula I

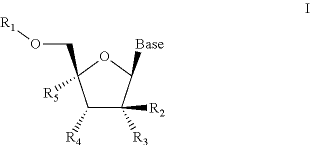

wherein the variables R₁, R₂, R₃, R₄, R₅, and Base are defined as described hereinabove.

### CROSS-REFERENCE TO RELATED APPLICATIONS

This application claims priority to U.S. provisional application Ser. No. 62/296,024, filed Feb. 16, 2016, the disclosure of which is incorporated herein by reference and is commonly owned.

### FIELD OF THE INVENTION

The invention relates to nucleoside derivatives as inhibitors of Zika Virus (ZIKV) replication. In particular, the invention is concerned with the use of 4′-azauridine (4AU) nucleoside derivatives as inhibitors of replication of Zika and related negative strand and positive strand RNA viruses and pharmaceutical compositions containing such compounds.

### BACKGROUND OF THE INVENTION

Zika Virus (ZIKV) is a member of the Flaviviridae family which are enveloped, icosahedral, nonsegmented, single-stranded, positive-sense RNA viruses whose human pathogens also include Dengue virus (DENV), West Nile virus (WNV), yellow fever virus (YFV), Japanese encephalitis virus (JEV), and tick-borne encephalitis virus (TBEV) among others.

In humans, Zika Virus initially causes a mild illness known as Zika fever, or Zika, which had been known to occur in tropical regions since the 1950s, but had been isolated to a narrow tropical equatorial region from Africa to Asia. However, by 2014, the virus had emerged on Pacific Islands and appeared to spread eastward from there. By 2015, the virus had spread to Central America and the Caribbean. Zika Virus was reported in May 2015 in South America, where the Zika outbreak has reached pandemic levels, and since then has spread throughout the Americas.

Once a person is infected, the incubation period for the virus is approximately 3-12 days. Symptoms of the disease are non-specific but may include fever, rash, joint pain, and conjunctivitis. It appears that only about 1 in 5 infected individuals will exhibit these symptoms and most will have mild symptoms.

Zika virus is an emerging mosquito-borne virus that has been associated with a large increase in the incidence of Guillain-Barre syndrome and severe pregnancy complications, including pregnancy loss, intrauterine growth restriction, eye defects, congenital brain abnormalities, microcephaly and other fetal abnormalities. Zika virus has been isolated from a fetal brain with microcephaly and virus particles could also be visualized by microscopy in that case. The recent very rapid spread of ZIKV through Brazil, other South American countries to the Caribbean (Puerto Rico), the large increase in travel associated Zika cases in the United States, and increasing numbers of case reports of sexual transmission reflect the substantial emerging global health burden of ZIKV-associated disease and the requirement for epidemiologic monitoring, risk and patient management in countries within but also outside of the Aedes mosquito geographic range. Zika virus can infect different cell types in humans, including dendritic cells, fibroblasts, keratinocytes and neurons, which contributes to immune evasion, neuronal damage and facilitation of virus spread. Zika virus has spread very rapidly since the first report of autochthonous transmission in Brazil in 2015. Epidemiologic and genetic analyses suggest that the virus is evolving with a rate of ˜1×10⁻³ substitutions per site per year. This is a mutation rate similar to that observed with other RNA viruses, such as Ebolavirus or HCV. Although the specific mutation rates may vary significantly depending on the source virus strain and host environment, the continuous sequence evolution of ZIKV poses a significant threat to human health, as virus variants with improved human adaptation, increased pathogenicity and increased transmissibility may emerge from the ongoing genetic drift. For example, the ZIKV sequence analysis of French Polynesian and American strains that are associated with reports of Guillain-Barré syndrome and microcephaly highlighted specific sequence variants that may be associated with disease complications and therefore have been proposed for further investigation.

The currently ongoing ZIKV outbreak in South America, the ongoing spread through Aedes mosquito transmission and human travel, the possibility of sexual transmission, and the high risk of neuronal and pregnancy complications translate into an urgent need for effective prophylaxis and treatment options to prevent and treat ZIKV infection and that can be used in early and advanced disease stages and also as pre-exposure and post-exposure prophylaxis by those at high risk of infection. Moreover, it has been reported that Aedes mosquito transmitted epizootics tended to follow Aedes mosquito transmitted Chikungunya virus epizootics and epidemics, and both viruses may circulate at the same time. It could therefore be advantageous to have a treatment and prophylaxis option that could be efficacious against both Zika and Chikungunya virus.

Currently, there is no vaccine or treatment for Zika Virus infection. Thus, the development of treatments for Zika Virus is a critical unmet need. There is a clear urgency to develop effective therapeutics for treatment of Zika Virus. Specifically, there is a need to develop therapeutics and compounds that selectively inhibit Zika viral replication that are useful for preventing Zika Virus infection in general and, critically, in pregnant women, and treating Zika-infected patients. The delivery of an efficacious treatment for ZIKV infection would be a groundbreaking innovation that would change the lives of many people with or at risk of ZIKV infection. Thus, the discovery of potent nucleoside inhibitors of ZIKV replication presents a highly unmet need in the field.

### SUMMARY OF THE INVENTION

The ideal treatment for ZIKV should be simple to administer, fast acting, efficacious (protection from pregnancy complications, resolution of symptoms and reduction of virus load) and well tolerated with minimal or no side effects. It should be simple to ship from manufacturing sites to health care providers and stable under typical tropical conditions of high temperature and humidity. Importantly, it should be broadly active across existing and future sequence variants, and it should show a high barrier to resistance to avoid the selection and transmission of drug resistant variants during a disease outbreak. Herein disclosed are nucleoside analogues that have been developed to exhibit these therapeutic properties in the treatment of Zika Virus.

The ZIKV inhibitors disclosed herein represent the first described nucleoside analogs with potent anti-ZIKV activity. These prodrugs introduced highly innovative monophosphate prodrug delivery technology to improve an already clinically tested nucleoside analog, 4AU, (4′-azidouridine). These compounds are ideally suited to provide the first effective options for prevention and treatment of ZIKV infection.

### The Compounds of Formula I as ZIKV Inhibitors

The compounds of Formula I are useful for the treatment of diseases mediated by Zika Virus and related RNA viruses and for pharmaceutical compositions comprising such compounds.

The application provides a method for preventing or treating Zika Virus comprising administering to a patient in need thereof a compound of Formula I

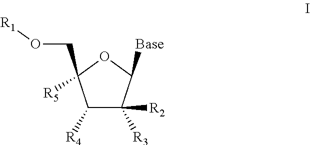

wherein:
R₁ is hydrogen, ethyl ester, or monophosphate, diphosphate, triphosphate, or phosphoramidate;

### R₂ is F, Me, or H;

### R₃ is OH, F, or H;

### R₄ is OH, F, or H;

R₅ is azido; and
Base is uracil, optionally substituted with methyl or Br;
or pharmaceutically acceptable salts thereof.

### BRIEF DESCRIPTION OF THE DRAWINGS

For a more complete understanding of the present disclosure and its advantages, reference is now made to the following descriptions, taken in conjunction with the accompanying drawings, in which:

FIG. 1. Graph of % infection of ZIK-V-RLuc at 72 h.p.i. vs. IC50 concentration (μM) of compound I-36.

FIG. 2. Graph of % infection of ZIK-V-RLuc at 72 h.p.i. vs. IC50 concentration (μM) of compound I-37.

FIG. 3. Antiviral activity of I-3 against ZIKV and CHIKV replication and cell viability determined in Huh-7 cell derived CHIKV replicon cells by intracellular ATP quantification (CellTiter-Glo). Mean values shown as individual data points; standard deviations as error bars.

FIG. 4. Mitochondrial toxicity in HepG2 cells after 11 days incubation with I-1 and I-35. Mitochondrial toxicity is apparent as a reduction in mitochondrially encoded protein COX-I as compared to nuclear encoded protein SDH-A. A decrease of the ratio between these two proteins indicates mitochondrial toxicity as shown by reference compounds chloramphenicol and 2′,3′-dideoxy-cytidine.

### DETAILED DESCRIPTION OF THE INVENTION

The application discloses improvements in scientific knowledge with respect to the treatment of Zika Virus by demonstrating an effective ribose modification that is capable of conferring potent antiviral activity against the ZIKV polymerase and likely broadly within the Flaviviridae family. Demonstrated is an improved understanding of how to design a nucleoside analog capable of targeting the viral polymerase, while avoiding cytotoxicity and mitochondrial toxicity which is critical knowledge to yield an effective and rational anti-ZIKV therapeutic. The application presents Zika Virus inhibitors which may greatly improve clinical practice in the management and prevention of ZIKV disease, as the nucleosides disclosed in this application can be used as a prophylactic for individuals at risk of exposure to ZIKV, as a post-exposure prophylaxis or as a treatment to rapidly abolish viral replication and disease progression in already infected individuals.

The instantly disclosed 4 AU prodrugs, representing effective ZIKV nucleoside inhibitors, have several commercially favorable features: 1) The antiviral potencies of the tested tested prodrugs are in the same range or more potent as compared to successful antiviral products (e.g. Tenofovir, Lamivudine, Abacavir, Ganciclovir); 2) the development of the disclosed prodrugs was intended to improve systemic delivery of nucleoside 4AU, which itself has already demonstrated preclinical safety in chronic toxicity studies in rats and monkeys and a safety profile in humans supportive of further development; 3) 4AU has demonstrated lack of genotoxicity; 4) the chemistry of the prodrugs has already been successfully used in antiviral products that are targeting multiple human cell types (Tenofovir alafenamide, Sofosbuvir); and 5) physicochemical properties of the prodrugs support the development into oral or intravenous treatments. Thus, the disclosed prodrugs' properties support their potential to achieve the ideal target profile for a successful product to treat ZIKV infections as described above.

The ZIKV inhibitors disclosed herein are nucleoside analogs with potent antiviral activity and selectivity and safety profiles to allow development of safe and efficacious treatments of viral diseases caused by RNA viruses; the prodrugs herein disclosed show potent activity against ZIKV. Notably, parent nucleoside 4AU was previously developed by Roche as a potential treatment of HCV infection and has completed chronic (13-week) toxicity studies in rats and dogs and a Phase 1 study in healthy volunteers and HCV patients. Despite showing antiviral efficacy and safety in HCV patients, 4AU was not further developed for the HCV indication when Sofosbuvir (another uridine nucleoside analog, but delivered as a monophosphate prodrug) showed higher antiviral activity against HCV infection. However, as disclosed herein, it was found that 4AUTP is also active against other flavivirus polymerases, and monophosphate prodrugs of 4AU provide potent antiviral activity against ZIKV infection. The ZIKV inhibitors disclosed herein have the potential to be efficacious against diseases caused by ZIKV, other flaviviruses and Chikungunya virus.

The ZIKV inhibitors disclosed herein are prodrugs of the nucleoside analog 4AU. 4AU is a nucleoside analog with intrinsic activity against flavivirus polymerases. 4AU has demonstrated preclinical safety in chronic toxicity studies and clinical safety in a Phase 1 human study. The prodrugs disclosed herein have been developed to deliver 4AU to multiple human cell types in a form where it can be rapidly converted to its active triphosphate species and therefore have the potential to inhibit ZIKV replication systemically. As disclosed herein, data have been generated to show that 4AU prodrugs could improve antiviral activity of 4AU more than 1000-fold. The development pathway for the prodrugs disclosed herein comprised use of panels of human cells and animal species that are biologically best suited to predict the pharmacology of a nucleotide (i.e. development is directed based on innovative knowledge learnings that demonstrate that the formation of active nucleoside triphosphate is the most important parameter to predict safety and efficacy profiles of antiviral nucleoside analogs), which can be different from classical small molecule development. Resulting from years of acquired expertise in nucleoside design and biology of 4AU, the prodrugs developed herein are preclinical assets that likely need no additional modifications to become successful clinical candidates. Unlike vaccines, therapeutic antibodies, or typical non-nucleoside small molecules, our novel potent ZIKV nucleoside analogs are expected to be usable as a prophylactic and therapeutic treatment, with intravenous (i.v.) or oral administration options, and with a high margin of safety. The current disclosure establishes the potential of the first nucleoside analog that inhibits ZIKV replication in human cells to become the best treatment and prophylaxis option for ZIKV.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:

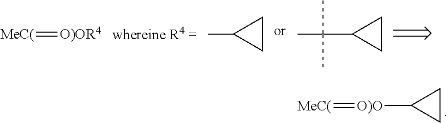

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “aryl alkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl,” as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiazoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkyl sulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl,” “heterocycloalkyl,” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkyl sulfonyl, aryl sulfonyl, alkylaminosulfonyl, arylaminosulfonyl, alkylsulfonylamino, arylsulfonylamino, alkylaminocarbonyl, aryl aminocarbonyl, alkylcarbonylamino, aryl carbonyl amino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of Zika Virus

The application provides a method for treating Zika Virus comprising administering to a patient in need thereof a compound selected from the group consisting of:
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
tert-butyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
(2R,3S,4R,5R)-2-azido-2-((((((S)-1-(benzyloxy)-1-oxopropan-2-yl)amino)(naphthalen-1-yloxy)phosphoryl)oxy)methyl)-5-(2,4-dioxo-3,4-dihydropyrimidin-1(2H)-yl)tetrahydrofuran-3,4-diyl dipropionate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((4-methoxynaphthalen-1-yl)oxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-leucinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-leucinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((l-bromonaphthalen-2-yl)oxy)phosphoryl)-L-leucinate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)glycinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((3-methoxynaphthalen-2-yl)oxy)phosphoryl)-L-alaninate;
ethyl N-((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-O-methyl-L-homoserinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-valinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(4-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(2-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(3-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(4-cyanophenoxy)phosphoryl)-L-alaninate;
isopropyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl 1-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)cyclopentane-1-carboxylate;
isopropyl 1-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)cyclopentane-1-carboxylate;
ethyl 2-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)-2-methylpropanoate;
isopropyl 2-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)-2-methylpropanoate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(3-cyanophenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(2-cyanophenoxy)phosphoryl)-L-alaninate;
(2R,3S,4R,5R)-2-azido-2-((((((S)-1-(tert-butoxy)-1-oxopropan-2-yl)amino)(naphthalen-1-yloxy)phosphoryl)oxy)methyl)-5-(2,4-dioxo-3,4-dihydropyrimidin-1(2H)-yl)tetrahydrofuran-3,4-diyl dipropionate;
(2R,3S,4R,5R)-2-azido-2-((((((S)-1-(tert-butoxy)-1-oxopropan-2-yl)amino)(phenoxy)phosphoryl)oxy)methyl)-5-(2,4-dioxo-3,4-dihydropyrimidin-1(2H)-yl)tetrahydrofuran-3,4-diyl dipropionate;
2,2,2-trifluoroethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
2,2,2-trifluoroethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(phenoxy)phosphoryl)-L-alaninate;
benzyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
tert-butyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
tert-butyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(naphthalen-1-yloxy)phosphoryl)-L-alaninate;
isopropyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
isopropyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(naphthalen-1-yloxy)phosphoryl)-L-alaninate;
ethyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate;
benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate; and
((((2R,3S,4R,5R)-2-azido-5-(2,4-dioxo-3,4-dihydropyrimidin-1(2H)-yl)-3,4-dihydroxytetrahydrofuran-2-yl)methoxy)(hydroxy)phosphoryl)-L-alanine;

or a pharmaceutically acceptable salt thereof.

The application provides a method of preventing or treating Zika Virus by administering to a patient in need thereof a composition comprising any one of the compounds listed above.

The application provides a method for preventing or treating Zika Virus comprising administering to a patient in need thereof a compound of Formula I:

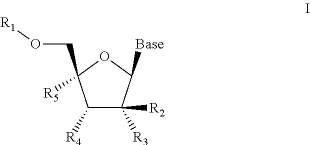

wherein:
R₁ is hydrogen, ethyl ester, or monophosphate, diphosphate, triphosphate, or phosphoramidate;

### R₂ is F, Me, or H;

### R₃ is OH, F, or H;

### R₄ is OH, F, or H;

R₅ is azido; and
Base is uracil, optionally substituted with methyl or Br;
or pharmaceutically acceptable salts thereof.

The application provides any one of the above methods for preventing or treating Zika Virus, further comprising administering at least one other antiviral agent.

The application provides a method of preventing or treating Zika Virus by administering to a patient in need thereof a composition comprising the compound of Formula 1.

The application provides a method of preventing or treating Zika Virus by administering to a patient in need thereof a composition comprising the compound of Formula I, admixed with a diluent.

The application provides a method of preventing or treating Zika Virus by administering to a patient in need thereof a composition comprising the speciated compounds listed above, admixed with a diluent.

The application provides a method of preventing or treating Zika Virus by administering to a patient in need thereof a composition comprising the compound of Formula I, further comprising administering at least one other antiviral agent.

The application provides a method of preventing or treating Zika Virus by administering to a patient in need thereof a composition comprising any one of the compounds speciated above, further comprising administering at least one other antiviral agent.

The application provides any of the above methods, further comprising administering at least one other antiviral agent.

The application provides a method of preventing or treating Zika Virus by administering to a patient in need thereof a compound of Formula I, wherein the compound is selected from the group consisting of:
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate; and
benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;

or a pharmaceutically acceptable salt thereof.

The application provides a method of preventing or treating Zika Virus by administering to a patient in need thereof a compound of Formula I, wherein the compound is benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

The application provides a method of preventing or treating Zika Virus by administering to a patient in need thereof a compound of Formula I, wherein the compound is ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

The application provides a method of preventing or treating Zika Virus by administering to a patient in need thereof a compound of Formula I, wherein the compound is benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

The application provides a method of preventing or treating Zika Virus by administering to a patient in need thereof a compound of Formula I, wherein the compound is benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

The application provides the use of any compound of Formula I for the preparation of a medicament for use in the treatment or prevention of Zika Virus.

The application provides the use of any one of the compounds speciated above for the preparation of a medicament for use in the treatment or prevention of Zika Virus.

The application provides for any of the embodiments of the invention herein described.

### Compounds for Method of Treating Zika Virus

The following representative compounds of generic formula I useful for the treatment of Zika Virus, as disclosed herein, are provided in the following Table I. The examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formula I.

TABLE I

| Compound | Nomenclature | Structure |
|---|---|---|
| I-1 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 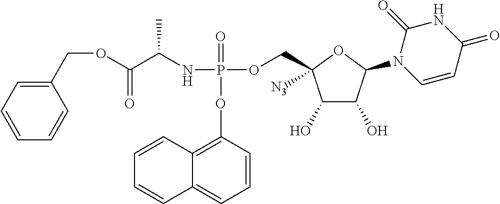 [SMILES: C[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OCc1ccccc1] |
| I-2 | tert-butyl ((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 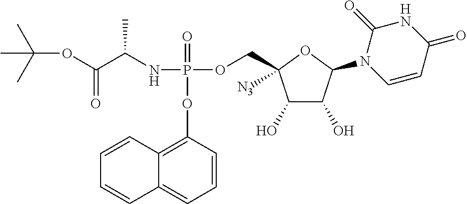 [SMILES: C[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OC(C)(C)C] |
| I-3 | ethyl ((((2R,3S,4R,5R)-2-azido- 5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 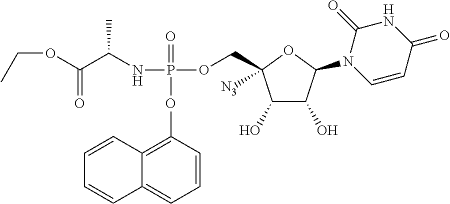 [SMILES: CCOC(=O)[C@H](C)NP(=O)(OC[C@@]1(N=[N+]=[N-])O[C@@H](n2ccc(=O)[nH]c2=O)[C@H](O)[C@@H]1O)Oc1cccc2ccccc12] |
| I-4 | (2R,3S,4R,5R)-2-azido-2- ((((((S)-1-(benzyloxy)-1- oxopropan-2- yl)amino)(naphthalen-1- yloxy)phosphoiyl)oxy)methyl)- 5-(2,4-dioxo-3,4- dihydropyrimidin-1(2H)- yl)tetrahydrofuran-3,4-diyl dipropionate | 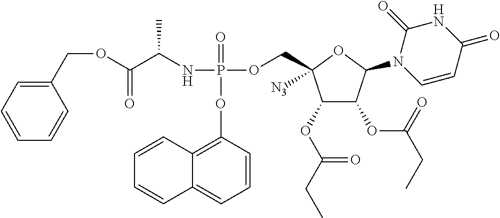 [SMILES: CCC(=O)O[C@H]1[C@H](n2ccc(=O)[nH]c2=O)O[C@@](COP(=O)(N[C@@H](C)C(=O)OCc2ccccc2)Oc2cccc3ccccc23)(N=[N+]=[N-])[C@H]1OC(=O)CC] |
| I-5 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)((4- methoxynaphthalen-1- yl)oxy)phosphoryl)-L-alaninate | 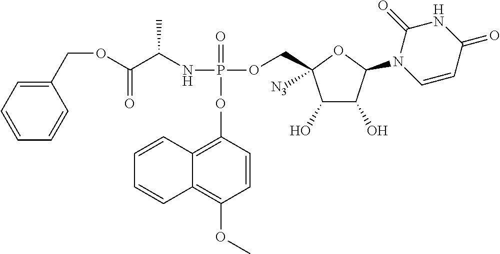 [SMILES: COc1ccc(OP(=O)(N[C@@H](C)C(=O)OCc2ccccc2)OC[C@@]2(N=[N+]=[N-])O[C@@H](n3ccc(=O)[nH]c3=O)[C@H](O)[C@@H]2O)c2ccccc12] |
| I-6 | ethyl ((((2R,3S,4R,5R)-2-ozido- 5-(2,4-dioxo-3,4- dihydropyrimidin-1(2H)-yl)-3,4- dihydroxytetrahydrofuran-2- yl)methoxy)(naphthalen-1- yloxy)phosphoryl)-L-leucinate | 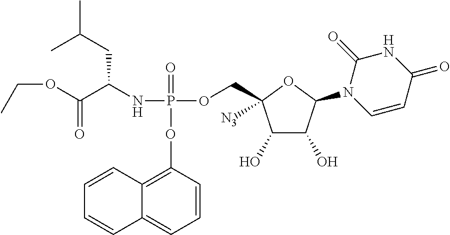 [SMILES: CCOC(=O)[C@H](CC(C)C)NP(=O)(OC[C@@]1(N=[N+]=[N-])O[C@@H](n2ccc(=O)[nH]c2=O)[C@H](O)[C@@H]1O)Oc1cccc2ccccc12] |
| I-7 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-leucinate | 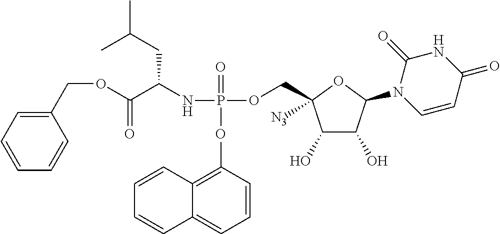 [SMILES: CC(C)C[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OCc1ccccc1] |
| I-8 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)((1- bromonaphthalen-2- yl)oxy)phosphoryl)-L-leucinate | 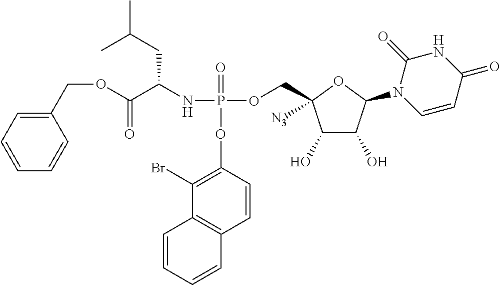 [SMILES: CC(C)C[C@H](NP(=O)(OC[C@@]1(N=[N+]=[N-])O[C@@H](n2ccc(=O)[nH]c2=O)[C@H](O)[C@@H]1O)Oc1ccc2ccccc2c1Br)C(=O)OCc1ccccc1] |
| I-9 | ethyl ((((2R,3S,4R,5R)-2-azido- 5-(2,4-dioxo-3,4- dihydropyrimidin-1(2H)-yl)-3,4- dihydroxytetrahydrofuran-2- yl)methoxy)(naphthalen-1- yloxy)phosphoryl)glycinate | 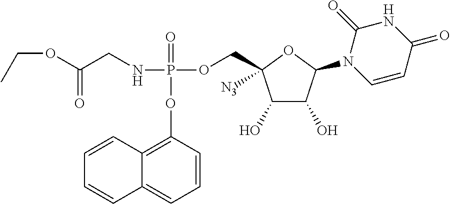 [SMILES: CCOC(=O)CNP(=O)(OC[C@@]1(N=[N+]=[N-])O[C@@H](n2ccc(=O)[nH]c2=O)[C@H](O)[C@@H]1O)Oc1cccc2ccccc12] |
| I-10 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)((3- methoxynaphthalen-2- yl)oxy)phosphoryl)-L-alaninate | 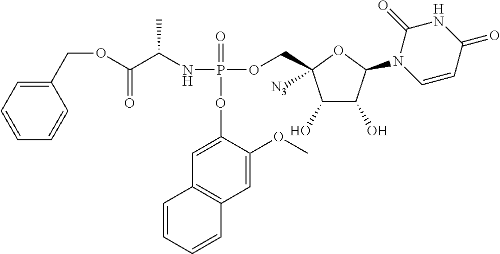 [SMILES: COc1cc2ccccc2cc1OP(=O)(N[C@@H](C)C(=O)OCc1ccccc1)OC[C@@]1(N=[N+]=[N-])O[C@@H](n2ccc(=O)[nH]c2=O)[C@H](O)[C@@H]1O] |
| I-11 | ethyl N-((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-O-methyl-L- homoserinate | 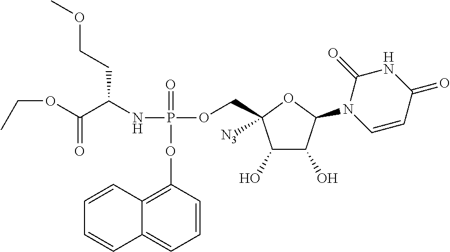 [SMILES: CCOC(=O)[C@H](CCOC)NP(=O)(OC[C@@]1(N=[N+]=[N-])O[C@@H](n2ccc(=O)[nH]c2=O)[C@H](O)[C@@H]1O)Oc1cccc2ccccc12] |
| I-12 | benzyl ((((2R,3S,4R,5R)-2- azido-5-(2,4-dioxo-3,4- dihydropyrimidin-1(2H)-yl)-3,4- dihydroxyetrahydrofuran-2- yl)methoxy)(naphthalen-1- yloxy)phosphoryl)-L-valinate | 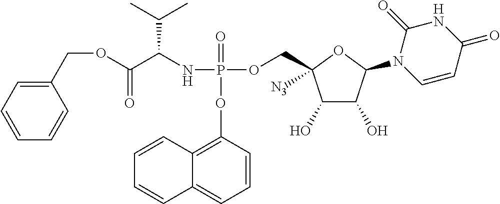 [SMILES: CC(C)[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OCc1ccccc1] |
| I-13 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(4- (trifluoromethyl)phenoxy) phosphoryl)-L-alaninate | 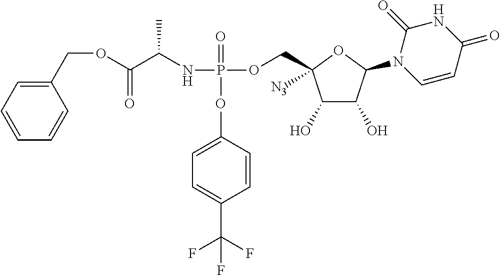 [SMILES: C[C@H](NP(=O)(OC[C@@]1(N=[N+]=[N-])O[C@@H](n2ccc(=O)[nH]c2=O)[C@H](O)[C@@H]1O)Oc1ccc(C(F)(F)F)cc1)C(=O)OCc1ccccc1] |
| I-14 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(2- (trifluoromethyl)phenoxy) phosphoryl)-L-alaninate | 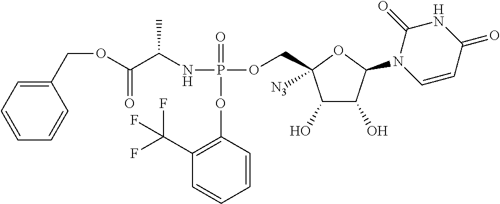 [SMILES: C[C@H](NP(=O)(OC[C@@]1(N=[N+]=[N-])O[C@@H](n2ccc(=O)[nH]c2=O)[C@H](O)[C@@H]1O)Oc1ccccc1C(F)(F)F)C(=O)OCc1ccccc1] |
| I-15 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(3- (trifluoromethyl)phenoxy) phosphoryl)-L-alaninate | 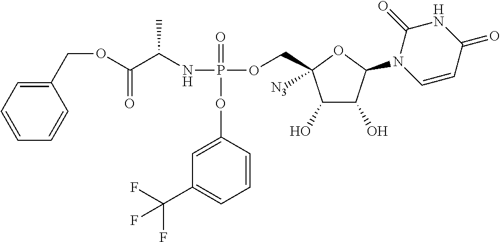 [SMILES: C[C@H](NP(=O)(OC[C@@]1(N=[N+]=[N-])O[C@@H](n2ccc(=O)[nH]c2=O)[C@H](O)[C@@H]1O)Oc1cccc(C(F)(F)F)c1)C(=O)OCc1ccccc1] |
| I-16 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(4- cyanophenoxy)phosphoryl)-L- alaninate | 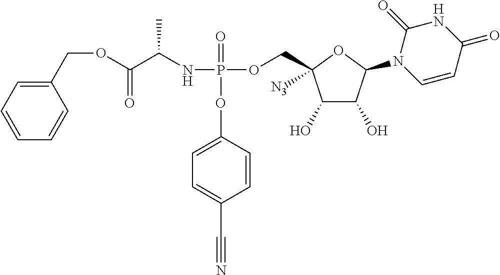 [SMILES: C[C@H](NP(=O)(OC[C@@]1(N=[N+]=[N-])O[C@@H](n2ccc(=O)[nH]c2=O)[C@H](O)[C@@H]1O)Oc1ccc(C#N)cc1)C(=O)OCc1ccccc1] |
| I-17 | isopropyl ((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 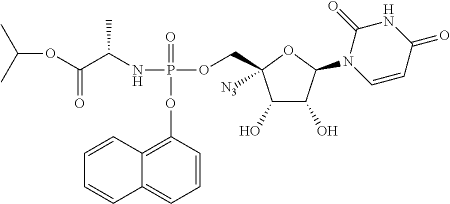 [SMILES: CC(C)OC(=O)[C@H](C)NP(=O)(OC[C@@]1(N=[N+]=[N-])O[C@@H](n2ccc(=O)[nH]c2=O)[C@H](O)[C@@H]1O)Oc1cccc2ccccc12] |
| I-18 | benzyl 1-(((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)animo) cyclopentane-1-carboxylate | 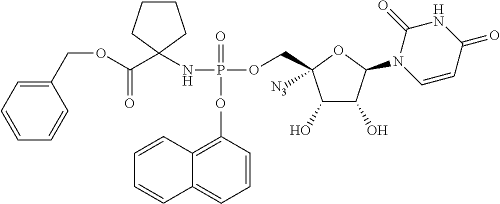 [SMILES: [N-]=[N+]=N[C@]1(COP(=O)(NC2(C(=O)OCc3ccccc3)CCCC2)Oc2cccc3ccccc23)O[C@@H](n2ccc(=O)[nH]c2=O)[C@H](O)[C@@H]1O] |
| I-19 | isopropyl 1-(((((2R,3S,4R,5R)- 2-azido-5-(2,4-dioxo-3,4- dihydropyrimidin-1(2H)-yl)-3,4- dihydroxytetrahydrofuran-2- yl)methoxy)(naphthalen-1- yloxy)phosphoryl)amino) cyclopentane-1-carboxylate | 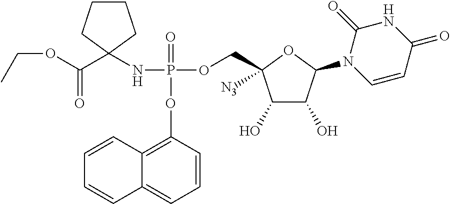 [SMILES: CCOC(=O)C1(NP(=O)(OC[C@@]2(N=[N+]=[N-])O[C@@H](n3ccc(=O)[nH]c3=O)[C@H](O)[C@@H]2O)Oc2cccc3ccccc23)CCCC1] |
| I-20 | ethyl 2-(((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)amino)-2- methylpropanoate | 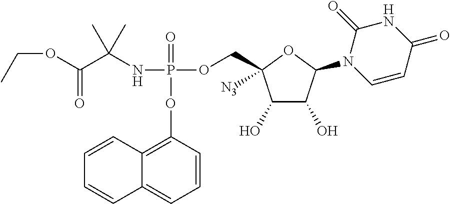 [SMILES: CCOC(=O)C(C)(C)NP(=O)(OC[C@@]1(N=[N+]=[N-])O[C@@H](n2ccc(=O)[nH]c2=O)[C@H](O)[C@@H]1O)Oc1cccc2ccccc12] |
| I-21 | isopropyl 2-(((((2R,3S,4R,5R)- 2-azido-5-(2,4-dioxo-3,4- dihydropyrimidin-1(2H)-yl)-3,4- dihydroxytetrahydrofuran-2- yl)methoxy)(naphthalen-1- yloxy)phosphoryl)amino)-2- methylpropanoate | 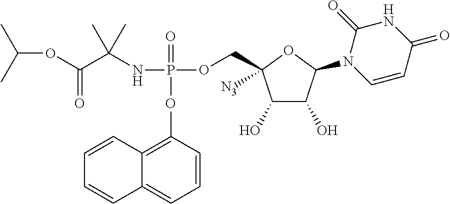 [SMILES: CC(C)OC(=O)C(C)(C)NP(=O)(OC[C@@]1(N=[N+]=[N-])O[C@@H](n2ccc(=O)[nH]c2=O)[C@H](O)[C@@H]1O)Oc1cccc2ccccc12] |
| I-22 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(3- cyanophenoxy)phosphoryl)-L- alanmate | 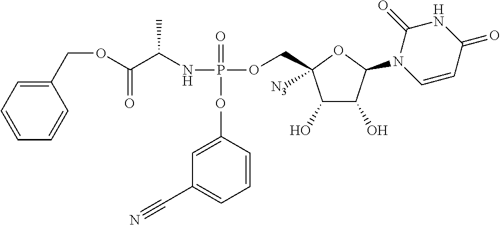 [SMILES: C[C@H](NP(=O)(OC[C@@]1(N=[N+]=[N-])O[C@@H](n2ccc(=O)[nH]c2=O)[C@H](O)[C@@H]1O)Oc1cccc(C#N)c1)C(=O)OCc1ccccc1] |
| I-23 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(2- cyanophenoxy)phosphoryl)-L- alaninate | 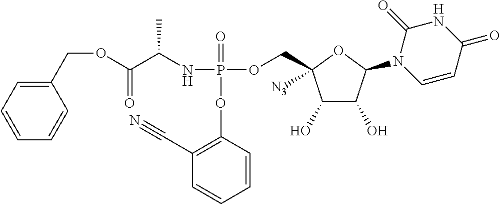 [SMILES: C[C@H](NP(=O)(OC[C@@]1(N=[N+]=[N-])O[C@@H](n2ccc(=O)[nH]c2=O)[C@H](O)[C@@H]1O)Oc1ccccc1C#N)C(=O)OCc1ccccc1] |
| I-24 | (2R,3S,4R,5R)-2-azido-2- ((((((S)-1-(tert-butoxy)-1- oxopropan-2- yl)amino)(naphthalen-1- yloxy)phosphoryl)oxy)methyl)- 5-(2,4-dioxo-3,4- dihydropyrimidin-1(2H)- yl)tetrahydrofuran-3,4-diyl dipropionate | 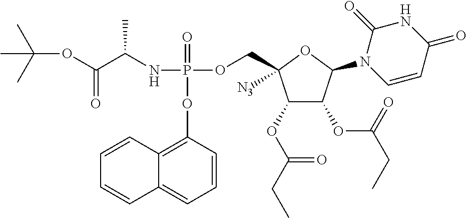 [SMILES: CCC(=O)O[C@H]1[C@H](n2ccc(=O)[nH]c2=O)O[C@@](COP(=O)(N[C@@H](C)C(=O)OC(C)(C)C)Oc2cccc3ccccc23)(N=[N+]=[N-])[C@H]1OC(=O)CC] |
| I-25 | (2R,3S,4R,5R)-2-azido-2- ((((((S)-1-(tert-butoxy)-1- oxopropan-2- yl)amino)(phenoxy)phosphoryl) oxy)methyl)-5-(2,4-dioxo-3,4- dihydropyrimidin-1(2H)- yl)tetrahydrofuran-3,4-diyl dipropionate | 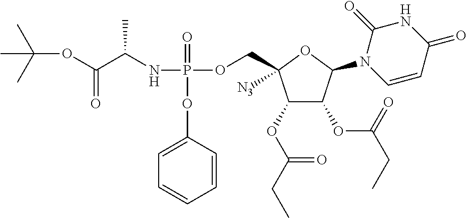 [SMILES: CCC(=O)O[C@H]1[C@H](n2ccc(=O)[nH]c2=O)O[C@@](COP(=O)(N[C@@H](C)C(=O)OC(C)(C)C)Oc2ccccc2)(N=[N+]=[N-])[C@H]1OC(=O)CC] |
| I-26 | 2,2,2-trifluoroethyl ((((2R,3S,4R,5R)-2-azido-5- (2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 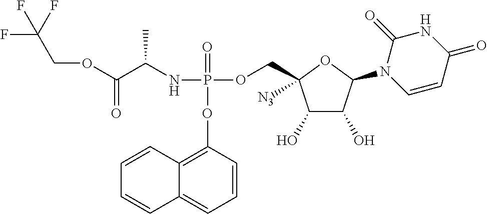 [SMILES: C[C@H](NP(=O)(OC[C@@]1(N=[N+]=[N-])O[C@@H](n2ccc(=O)[nH]c2=O)[C@H](O)[C@@H]1O)Oc1cccc2ccccc12)C(=O)OCC(F)(F)F] |
| I-27 | 2,2,2-trifluoroethyl ((((2R,3S,4R,5R)-2-azido-5- (2,4-dioxo-3,4- dihydropyrimidin-1(2H)-yl)-3,4- dihydroxytetrahydrofuran-2- yl)methoxy)(phenoxy) phosphoryl)-L-alanmate | 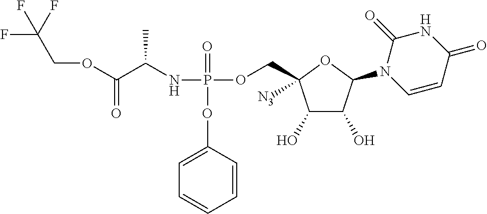 [SMILES: C[C@H](NP(=O)(OC[C@@]1(N=[N+]=[N-])O[C@@H](n2ccc(=O)[nH]c2=O)[C@H](O)[C@@H]1O)Oc1ccccc1)C(=O)OCC(F)(F)F] |
| I-28 | benzyl ((((2R,3S,4R,5R)-2- azido-3,4-dihydroxy-5-(5- methyl-2,4-dioxo-3,4- dihydropyrimidin-1(2H)- yl)tetrahydrofuran-2- yl)methoxy)(phenoxy) phosphoryl)-L-alaninate | 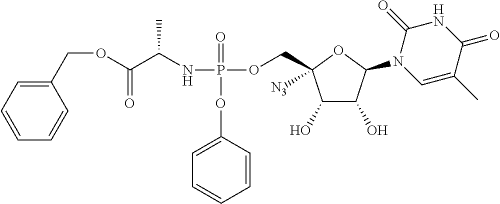 [SMILES: Cc1cn([C@@H]2O[C@@](COP(=O)(N[C@@H](C)C(=O)OCc3ccccc3)Oc3ccccc3)(N=[N+]=[N-])[C@@H](O)[C@H]2O)c(=O)[nH]c1=O] |
| I-29 | tert-butyl ((((2R,3S,4R,5R)-2- azido-3,4-dihydroxy-5-(5- methyl-2,4-dioxo-3,4- dihydropyrimidin-1(2H)- yl)tetrahydrofuran-2- yl)methoxy)(phenoxy) phosphoryl)-L-alaninate | 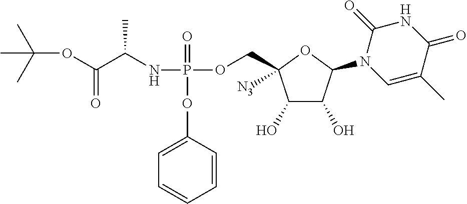 [SMILES: Cc1cn([C@@H]2O[C@@](COP(=O)(N[C@@H](C)C(=O)OC(C)(C)C)Oc3ccccc3)(N=[N+]=[N-])[C@@H](O)[C@H]2O)c(=O)[nH]c1=O] |
| I-30 | tert-butyl ((((2R,3S,4R,5R)-2- azido-3,4-dihydroxy-5-(5- methyl-2,4-dioxo-3,4- dihydropyrimidin-1(2H)- yl)tetrahydrofuran-2- yl)methoxy)(naphthalen-1- yloxy)phosphoryl)-L-alaninate | 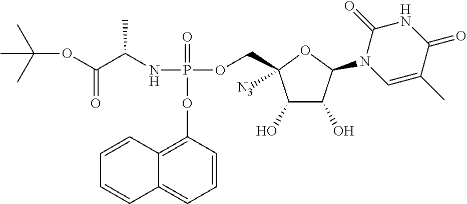 [SMILES: Cc1cn([C@@H]2O[C@@](COP(=O)(N[C@@H](C)C(=O)OC(C)(C)C)Oc3cccc4ccccc34)(N=[N+]=[N-])[C@@H](O)[C@H]2O)c(=O)[nH]c1=O] |
| I-31 | isopropyl ((((2R,3S,4R,5R)-2- azido-3,4-dihydroxy-5-(5- methyl-2,4-dioxo-3,4- dihydropyrimidin-1(2H)- yl)tetrahydrofuran-2- yl)methoxy)(phenoxy) phosphoryl)-L-alaninate | 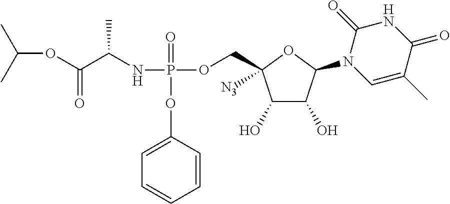 [SMILES: Cc1cn([C@@H]2O[C@@](COP(=O)(N[C@@H](C)C(=O)OC(C)C)Oc3ccccc3)(N=[N+]=[N-])[C@@H](O)[C@H]2O)c(=O)[nH]c1=O] |
| I-32 | isopropyl ((((2R,3S,4R,5R)-2- azido-3,4-dihydroxy-5-(5- methyl-2,4-dioxo-3,4- dihydropyrimidin-1(2H)- yl)tetrahydrofuran-2- yl)methoxy)(naphthalen-1- yloxy)phosphoryl)-L-alaninate | 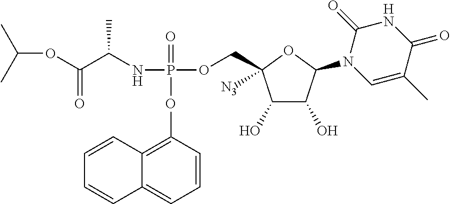 [SMILES: Cc1cn([C@@H]2O[C@@](COP(=O)(N[C@@H](C)C(=O)OC(C)C)Oc3cccc4ccccc34)(N=[N+]=[N-])[C@@H](O)[C@H]2O)c(=O)[nH]c1=O] |
| I-33 | ethyl ((((2R,3S,4R,5R)-2-azido- 3,4-dihydroxy-5-(5-methyl-2,4- dioxo-3,4-dihydropyrimidin- 1(2H)-yl)tetrahydrofuran-2- yl)methoxy)(phenoxy) phosphoryl)-L-alaninate | 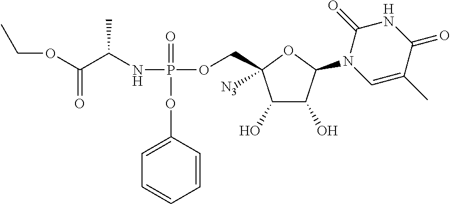 [SMILES: CCOC(=O)[C@H](C)NP(=O)(OC[C@@]1(N=[N+]=[N-])O[C@@H](n2cc(C)c(=O)[nH]c2=O)[C@H](O)[C@@H]1O)Oc1ccccc1] |
| I-34 | ethyl ((((2R,3S,4R,5R)-2-azido- 5-(2,4-dioxo-3,4- dihydropyrimidin-1(2H)-yl)-3,4- dihydroxytetrahydrofuran-2- yl)methoxy)(naphthalen-1- yloxy)phosphoryl)-L- phenylalaninate | 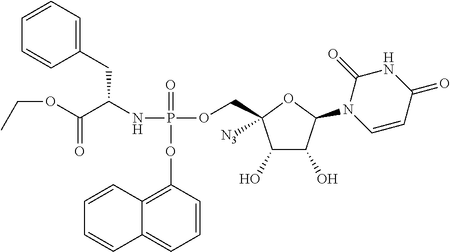 [SMILES: CCOC(=O)[C@H](Cc1ccccc1)NP(=O)(OC[C@@]1(N=[N+]=[N-])O[C@@H](n2ccc(=O)[nH]c2=O)[C@H](O)[C@@H]1O)Oc1cccc2ccccc12] |
| I-35 | benzyl ((((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L- phenylalaninate | 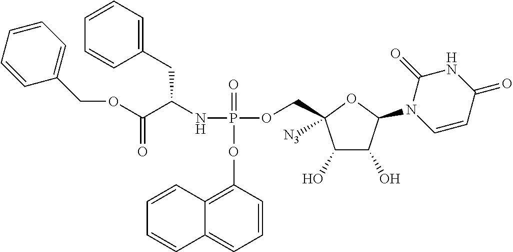 [SMILES: [N-]=[N+]=N[C@]1(COP(=O)(N[C@@H](Cc2ccccc2)C(=O)OCc2ccccc2)Oc2cccc3ccccc23)O[C@@H](n2ccc(=O)[nH]c2=O)[C@H](O)[C@@H]1O] |
| I-36 | benzyl ((R)-(((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 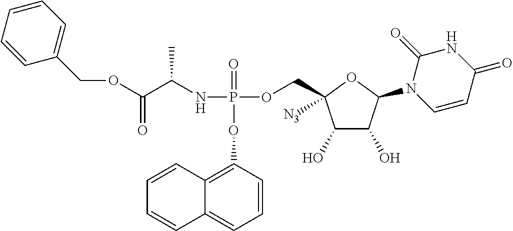 |
| I-37 | benzyl ((S)-(((2R,3S,4R,5R)-2- azido-5-(2,4-dioxo-3,4- dihydropyrimidin-1(2H)-yl)-3,4- dihydroxytetrahydrofuran-2- yl)methoxy)(naphthalen-1- yloxy)phosphoryl)-L-alaninate | 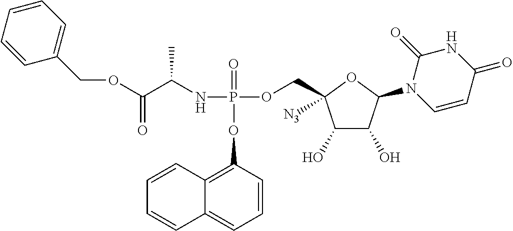 |
| I-38 | ((((2R,3S,4R,5R)-2-azido-5- (2,4-dioxo-3,4- dihydropyrimidin-1(2H)-yl)-3,4- dihydroxytetrahydrofuran-2- yl)methoxy)(hydroxy) phosphoryl)-L-alanine | 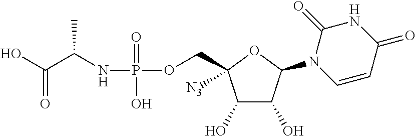 [SMILES: C[C@H](NP(=O)(O)OC[C@@]1(N=[N+]=[N-])O[C@@H](n2ccc(=O)[nH]c2=O)[C@H](O)[C@@H]1O)C(=O)O] |

### Pharmaceutical Compositions and Administration

Pharmaceutical compositions of the subject Compounds for administration via several routes were prepared as described in this Example.

### Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP | 1.0% |
| (polyvinylpyrrolidine) | |

The ingredients are combined and granulated using a solvent such as methanol. The formulation is then dried and formed into tablets (containing about 20 mg of active compound) with an appropriate tablet machine.

### Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

### Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Dosage and Administration:

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multidose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Method of Treatment

The compounds of the invention and their isomeric forms and pharmaceutically acceptable salts thereof are useful in treating and preventing Zika virus infection.

The application provides a method for treating a Zika virus infection comprising administering to a patient in need thereof a therapeutically effective amount of a compound of Formula I.

The application provides a method for inhibiting replication of Zika virus in a cell comprising administering a compound of Formula I.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), 2,3-Dichloro-5,6-dicyano-1,4-benzoquinone (DDQ), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), 2-ethoxy-1-ethoxycarbonyl-1,2-dihydroquinoline (EEDQ), ethyl (Et), ethyl acetate (EtOAc), ethanol (EtOH), 2-ethoxy-2H-quinoline-1-carboxylic acid ethyl ester (EEDQ), diethyl ether (Et₂O), ethyl isopropyl ether (EtOiPr), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), isopropylmagnesium chloride (iPrMgCl), hexamethyl disilazane (HMDS), liquid chromatography mass spectrometry (LCMS), lithium hexamethyl disilazane (LiHMDS), meta-chloroperoxybenzoic acid (m-CPBA), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), methyl tetrahydrofuran (MeTHF), N-bromosuccinimide (NBS), n-Butyllithium (nBuLi), N-carboxyanhydride (NCA), N-chlorosuccinimide (NCS), N-methylmorpholine (NMM), N-methylpyrrolidone (NMP), naphthyl (Np), pyridinium chlorochromate (PCC), Dichloro-((bis-diphenylphosphino)ferrocenyl) palladium(II) (Pd(dppf)Cl₂), palladium(II) acetate (Pd(OAc)₂), tris(dibenzylideneacetone)dipalladium(0) (Pd₂(dba)₃), pyridinium dichromate (PDC), phenyl (Ph), propyl (Pr), iso-propyl (i-Pr), pounds per square inch (psi), pyridine (pyr), 1,2,3,4,5-Pentaphenyl-1′-(di-tert-butylphosphino)ferrocene (Q-Phos), room temperature (ambient temperature, rt or RT), sec-Butyllithium (sBuLi), tert-butyldimethylsilyl or t-BuMe₂Si (TBDMS), tetra-n-butylammonium fluoride (TBAF), triethylamine (TEA or Et₃N), 2,2,6,6-tetramethylpiperidine 1-oxyl (TEMPO), triflate or CF₃SO₂-(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), and N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Conditions

Compounds of the invention can be made by a variety of methods depicted in the illustrative synthetic reactions described below in the Examples section. U.S. Pat. Nos. 7,608,599, and 6,846,810 disclose the preparation of antiviral nucleoside phosphoramidates, and 4′-azido cytidine phosphoramidates, respectively, and are herein incorporated by reference in their entireties. U.S. Pat. No. 8,236,779 discloses the preparation of antiviral nucleoside phosphoramidates of 4AU for the treatment of HCV and is herein incorporated by reference in its entirety.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### General Methodology

TLC was carried out on precoated, aluminum backed plates (60 F-54, 0.2 mm thickness; supplied by E. Merck AG, Darmstad, Germany) developed by ascending method. After solvent evaporation, compounds were detected by irradiation with an UV lamp at 254 nm or 366 nm observation of quenching of the fluorescence. Chromatography columns were slurry packed in the appropriate eluent under pressure, with silica gel, 60 A, 40-60 μm, Phase Sep, UK). Samples were applied as a concentrated solution in the same eluent, or pre-adsorbed on silica gel. ¹H and ¹³C NMR spectra were recorded on a Bruker Advance DPX300 spectrometer (300 MHz and 75 MHz respectively) and autocalibrated to the deuterated solvent reference peak. All ¹³C NMR were proton decoupled. The following abbreviations are used in the assignment of NMR signals: s (singlet), d (doublet), t (triplet), qu (quartet), q (quintet), m (multiplet), bs (broad signal), dd (double doublet), dt (double triplet). Low-resolution mass spectra were run on a VG Platform II Fisons instrument (atmospheric pressure ionization, electrospray mass spectrometry) in either negative or positive mode.

The solvents used were anhydrous and used as purchased from Aldrich. All glassware was oven dried at 130° C. for several hours and allowed to cool under a stream of dry nitrogen.

The compounds of formula I may be prepared by various methods known in the art of organic chemistry in general and nucleoside analogue synthesis in particular. The starting materials for the syntheses are either readily available from commercial sources or are known or may themselves be prepared by techniques known in the art. General reviews of the preparation of nucleoside analogues are included in the following publications:
A M Michelson “The Chemistry of Nucleosides and Nucleotides”, Academic Press, New York 1963.
L Goodman “Basic Principles in Nucleic Acid Chemistry” Ed P O P Ts'O, Academic Press, New York 1974, Vol. 1, chapter 2.
“Synthetic Procedures in Nucleic acid Chemistry” Ed W W Zorbach and R S Tipson, Wiley, New York, 1973, Vol. 1 and 2.
The synthesis of carbocylic nucleosides has been reviewed by L Agrofoglio et al, Tetrahedron, 1994, 50, 10611.

The strategies available for the synthesis of compounds of formula I include:

1. Modification or interconversion of preformed nucleosides; or

2. Construction of the heterocyclic base after glycosylation; or

3. Condensation of a protected furanose, thiofuranose or cyclopentane derivative with a pyrimidine (B2) base.

These methods will be further discussed below:

### Modification or Interconversion of Preformed Nucleosides.

Such methods include on the one hand modification of the 9-purinyl or 1-pyrimidyl residue or on the other hand modification of the carbohydrate moiety.

A. Modification of the Purinyl or Pyrimidyl Moiety:
1. The deamination of aminopurine or aminopyrimidine nucleosides as described by J. R. Tittensor and R. T. Walker European Polymer J., 1968, 4, 39 and H. Hayatsu, Progress in Nucleic Acid Research and Molecular Biology 1976, Vol. 16, p 75.
2. The conversion of the 4-hydroxy group of 4-hydroxypyrimidine nucleosides to a leaving group and displacement with nucleophilic reagents. Such leaving groups include halogen as described by J. Brokes and J. Beranek, Col. Czech. Chem. Comm., 1974, 39, 3100 or 1,2,4-triazole as described by K. J. Divakar and C. B. Reece, J. Chem. Soc. Perkin Trans. I, 1982, 1171.
3. 5-Substitution of pyrimidine nucleosides has been achieved by the use of 5-metallo derivatives such as 5-mercuri or 5-palladium for example as described by D. E. Bergstrom and J. L. Ruth J. Amer. Chem. Soc., 1976, 98, 1587. Introduction of fluoro into the 5 position of pyrimidine nucleosides can be achieved with reagents such as trifluoromethyl hypofluorite as described by M. J. Robins, Ann New York Acad. Sci. 1975, 255, 104.
4. Modified purine nucleosides may be prepared from the corresponding purine nucleoside derivatives wherein the 2, 6 or 8 substituent is a suitable leaving group such as halogen or sulphonate or 1,3,4-triazole. 6 substituted purine nucleosides may be prepared by treatment of the appropriate 6-halopurine or 6-(1,2,4-triazol-4-yl)-purine nucleoside derivatives with the appropriate nucleophilic reagent as described by V. Nair and A. J. Fassbender Tetrahedron, 1993, 49, 2169 and by V. Samano, R. W. Miles and M. J. Robins, J. Am. Chem. Soc., 1994, 116, 9331.
5. Similarly 8-substituted purine nucleosides can be prepared by treatment of the corresponding 8-halopurine nucleoside with the appropriate nucleophilic reagent as described by L. Tai-Shun, C. Jia-Chong, I. Kimiko and A. C. Sartorelli, J. Med. Chem., 1985, 28, 1481; Nandanan et al, J. Med. Chem., 1999, 42, 1625; J. Jansons, Y. Maurinsh, and M. Lidaks, Nucleosides Nucleotides, 1995, 14, 1709. Introduction of an 8-cyano substituent can be accomplished by displacement using a metal cyanide as described by L-L. Gundersen, Acta. Chem. Scand. 1996, 50, 58. 2-Modified purine nucleoside may be prepared in a similar fashion as described by T. Steinbrecher, C. Wamelung, F. Oesch and A. Seidl, Angew. Chem. Int. Ed. Engl., 1993, 32, 404.
6. Where the substituent at the 2 or 8-position of the purine nucleoside is linked via a carbon carbon bond e. g. alkyl, then metal catalysed cross-coupling procedures can be used starting with the appropriate 2 or 8-halosubstituted purine nucleoside analogue as described by A. A. Van Aerschott, et al, J. Med. Chem., 1993, 36, 2938; V. Nair and G. S. Buenger, J. Am. Chem. Soc., 1989, 111(22), 8502; C. Tu, C. Keane and B. E. Eaton Nucleosides Nucleotides, 1995, 14, 1631.

B. Modification of the Carbohydrate Moiety:
1. Following the introduction of protecting groups, which are compatible with the subsequent chemistry, azide may be introduced at the 4′-position by treatment of the 4′,5′-didehydro nucleoside with iodine azide as exemplified by H. Maag et al, J. Med. Chem., 1992, 35, 1440. An alkoxide may be introduced at the 4′-position by treatment of the 4′,5′-didehydro nucleoside with iodine followed by an alcohol and lead carbonate as exemplified by J. P. Verheyden and J. G. Moffatt, J. Am. Chem. Soc., 1975, 97(15), 4386. Fluoride may be introduced at the 4′-position by treatment of the 4′,5′-didehydro nucleoside with iodine followed by silver(I)fluoride as described by G. R. Owen et al, J. Org. Chem., 1976, 41(8), 3010 or A. Maguire et al, J. Chem. Soc. Perkin Trans. 1, 1993, 1(15), 1795. A 4′-formyl group can be introduced and subsequently converted to a wide range of substituents including but not limited to 4′-haloalkyl, 4′-ethynyl, 4′-oximinomethyl, and 4′-cyano as exemplified by M. Nomura et al., J. Med. Chem., 1999, 42, 2901.
2. Modification of either the 2′-hydroxy substituent or 3′-hydroxy substituent in the nucleoside analogue is possible.
3. Conversion of the 3-hydroxy to a leaving group such as halo by reaction with for example triphenyl phosphine and a tetrahaloalkane as described for example by L. De Napoli et al, Nucleosides Nucleotides, 1993, 12, 981, followed by reduction provides the 3-deoxysugar derivatives as described by D. G. Norman and C. B. Reese, Synthesis 1983, 304.
4. Derivatisation of the 3 hydroxy group by conversion to a triflate group followed by reduction using sodium borohydride as described by S. A. Surzhykov et al, Nucleosides Nucleotides, 1994, 13(10), 2283. Direct introduction of a fluorine substituent can be accomplished with fluorinating agents such as diethylamino [symbol] sulphur trifluoride as described by P. Herdewijn, A. Van Aerschot and L. Kerremans, NucleosidesNucleotides, 1989, 8, 65.
5. Conversion of the hydroxy substituent to a leaving group such as halo or sulphonate also allows displacement using nucleophilic reagents such as tetrabutylammonium fluoride, lithium azide, or metal cyanides as exemplified by H. Hrebabecky, A. Holy and E. de Clercq, Collect. Czech. Chem. Comm. 1990, 55, 1800; K. E. B. Parkes and K. Taylor, Tet. Lett., 1988, 29, 2995; H. M. Pfundheller et al, Helv. Chim. Acta, 2000, 83, 128.
6. Reaction of 2′-keto nucleosides with fluorinating agents such as diethylamino sulfur trifluoride can be used to prepare 2′,2′-difluoronucleosides as described by D. Bergstrom, E. Romo and P. Shum Nucleosides Nucleotides, 1987, 6, 53.
   Construction of the Heterocyclic Base after Glycosylation.
A. Those which for example utilise furanosylamine derivatives as described by N. J. Cusack, B. J. Hildick, D. H. Robinson, P. W. Rugg and G. Shaw J. Chem. Soc. Perkin Trans., I 1973, 1720 or G. Shaw, R. N. Warrener, M. H. Maguire and R. K. Ralph, J. Chem. Soc., 1958, 2294.
B. Those which utilise for example furanosylureas for pyrimidine nucleoside synthesis as described by J. \check{S}mejkal, J. Farkas, and F. \check{S}orm, Coll. Czech. Chem. Comm., 1966, 31, 291.
C. The preparation of purine nucleosides from imidazole nucleosides is reviewed by L. B. Townsend, Chem. Rev., 1967, 67, 533.
D. The preparation of compounds of formula I wherein X is CH2 can be accomplished from 1-hydroxymethyl-4-aminocyclopentane derivatives as described by Y. F. Shealy and J. D. Clayton J. Am. Chem. Soc., 1969, 91, 3075; R. Vince and S. Daluge J. Org. Chem., 1980, 45, 531; R. C. Cermak and R. Vince, Tet. Lett., 1981, 2331; R. D. Elliott et al, J. Med. Chem., 1994, 37, 739.
   Condensation of a Protected Furanose, Thiofuranose or Cyclopentane Derivative with a Pyrimidine Derivative.

The condensation reaction of a protected furanose, thiofuranose or cyclopentane derivative with an appropriate purine or pyrimidine derivative may be performed using standard methods including the use of a Lewis acid catalyst such as mercuric bromide or stannic chloride or trimethylsilyltrifluoromethane sulphonate in solvents such as acetonitrile, 1,2-dichloroethane, dichloromethane, chloroform or toluene at reduced, ambient or elevated temperature. Examples for the condensation reaction of a protected furanose or thiofuranose
with heavy metal derivatives of purines or pyrimidines derivatives (e. g. chloromercuri derivatives) are described by J Davoll and B. A. Lowry, J. Am. Chem. Soc., 1951, 73, 1650; J. J. Fox, N. Yung, J. Davoll and G. B. Brown, J. Am. Chem. Soc., 1956, 78, 2117.
with alkoxy pyrimidines are described by K. A. Watanabe, D. H. Hollenberg and J. J. Fox., Carbohydrates. Nucleosides and Nucleotides. 1974, 1, 1.
with silyl derivatives of purines or pyrimidines as described by U. Niedballa and H. Vorbruggen, J. Org. Chem., 1976, 41, 2084; U. Niedballa and H. Vorbruggen, J. Org. Chem., 1974, 39, 3672. A. J. Hubbard, A. S. Jones and R. T. Walker, Nucleic Acids Res., 1984, 12, 6827.

Furthermore:
the fusion of per-acylated sugars with purines under vacuum in the presence of p-toluene sulphonic acid has been described by T. Simadate, Y. Ishudo and T. Sato, Chem. Abs., 1962, 56, 11 692 and W. Pfleiderer, R. K. Robins, Chem. Ber. 1965, 98, 1511.
the condensation reactions have been described by K. A. Watanabe, D. H. Hollenberg and J. J. Fox, Carbohydrates Nucleosides and Nucleotides, 1974, 1, 1.

Examples for the condensation reaction of a protected cyclopentane derivative with an appropriate purine derivative or pyrimidine derivative are given in H. Kapeller, H. Baumgartner and H. Griengl, Monattsh Chem., 1997, 128, 191 and P. Wang et al, Tet. Lett., 1997, 38, 4207; or by T. Jenny et al. Helv. Chim. Acta, 1992, 25, 1944.

Such methods often result in mixtures of anomeric nucleoside derivatives which can be separated by standard techniques known to the art such as recrystallisation, column chromatography, high performance liquid chromatography or super critical fluid chromatography.

The pyrimidines derivatives for above condensation reactions can be obtained commercially or can be prepared by procedures known to the art.

The preparation of pyrimidines derivatives is reviewed by D. J. Brown in “The Chemistry of Heterocyclic Compounds—The Pyrimidines” 1962 and Supplement 1, 1970, pub John Wiley and Sons, New York, by D. J. Brown in “Comprehensive Heterocyclic Chemistry” pub Pergamon Press Vol. 5 chapter 4. 09, p 499 and by K. Unheim and T. Benneche in “Comprehensive Heterocyclic Chemistry II” pub Pergamon Press Vol. 6 chapter 6. 02 p 93.

Furanose derivatives can be prepared from commercially available carbohydrate starting materials such as the D forms of ribose, arabinose, xylose or lyxose, following introduction of protecting groups which are compatible with the chemistry.

4-Substituted furanoses with the substituent containing a carbon attached to the 4-position of the furanose, for example alkyl, alkenyl, alkynyl, haloalkyl, acyl, alkoxycarbonyl, hydroxyalkyl, alkoxyalkyl, cyano, oximinomethyl, alkoxyimino [symbol] methyl, alkylaminocarbonyl and acyl can be prepared from the corresponding 4-formyl furanose. The preparation of one such 4-formylfuranose is described by H. Ohrui et al., J. Med. Chem., 2000, 43, 5416. 4-Haloalkyl furanoses may be prepared from the corresponding 4-hydroxymethyl furanoses (e. g., K. Kitano et al, Tetrahedron, 1997, 53(39), 13315). 4-Methyl furanoses can be prepared by the method described by T. Waga et al, Biosci. Biotech. Biochem. 1993, 19(7), 408.

2,2-Difluorofuranose derivatives can be prepared from D-glucose or D-mannose as described by R. Fernandez, M. I. Mateu, R. Echarri and S. Castillon Tetrahedron, 1998, 54, 3523. The thiofuranose derivatives can be prepared by literature procedures such as L. Bellon, J. L. Barascut, J. L. Imbach, Nucleosides and Nucleotides 1992, 11, 1467 and modified in a similar fashion to the furanose analogues described above.

The cyclopentane derivatives can be prepared by methods known in the art of organic chemistry and by methods and references included in L. Agrofolio et al, Tetrahedron, 1994, 50, 10611.

The preformed nucleoside derivatives are either available commercially or synthesised in accordance with the methods described above.

### General Schemes:

U.S. Pat. No. 7,608,599 discloses the preparation of antiviral nucleoside phosphoramidates and is herein incorporated by reference in its entirety.

The invention also provides methods of treating diseases mediated by Hepatitis C virus by administering a compound of formula I. The compound can be administered alone or in combination with an immune system modulator, an antiviral agent, or an anti-inflammatory agent. The invention further includes compositions for the treatment of treating diseases mediated by Hepatitis C virus by administering a therapeutically effective amount of a compound of formula I.

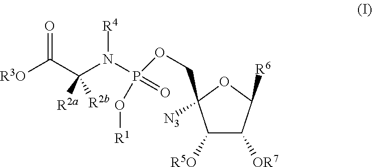

Compounds of the present invention are pro-drugs or bioprecursors of the parent nucleoside and are converted in vivo to the compound of formula I wherein R¹ is Ph or Np, optionally substituted with lower alkyl, lower alkoxy, hydroxy, halo, or halo lower alkyl; R^{2a}, R^{2b}, R³, R⁴, R⁵, and R⁷ are hydrogen. Prodrugs include acyl derivatives, amino acid esters, alkoxycarbonyl, aryloxycarbonyl, thioalkylcarbonyl and arylthiocarbonyl nucleoside or pharmaceutically acceptable salts thereof; and R⁶ is uracil.

One embodiment of the present invention is a nucleoside derivative according to formula I wherein R¹, R^{2a}, R^{2b}, R³, R⁴, R⁵, and R⁷ are independently selected from the groups defined hereinabove with the proviso that at least one of R^{2b}, R³, or R⁴ is other than hydrogen.

In another embodiment of the present invention there is provided a compound according to formula I wherein R¹ is Ph or Np, optionally substituted with lower alkyl, lower alkoxy, hydroxy, halo, or halo lower alkyl; R⁵ and R⁷ each are independently COR⁸, C(═O)OR⁸ or C(═O)SR⁸ and each R⁸ is independently selected from the group consisting of C₁₋₇ unbranched or branched lower alkyl, phenyl and CH₂OPh.

In another embodiment of the present invention there is provided a compound according to formula I wherein R¹ is Ph, optionally substituted with lower alkyl, lower alkoxy, hydroxy, halo, or halo lower alkyl; R⁵ and R⁷ are C(═O)R⁸ and each R⁸ is independently selected from the group consisting of C₁₋₇ unbranched or branched lower alkyl, phenyl and CH₂OPh.

In another embodiment of the present invention there is provided a compound according to formula I wherein R¹ is Ph, optionally substituted with lower alkyl, lower alkoxy, hydroxy, halo, or halo lower alkyl; R⁵ and R⁷ are COR⁸, C(═O)OR⁸, C(═O)SR⁸ or COCH(R⁸)NHR⁸; R^{2a}, R^{2b}, and R⁴ are hydrogen; R⁸ is selected from H or a group consisting of C₁₋₇ unbranched or branched lower alkyl, C₃₋₈ cycloalkyl, phenyl and the side chain of a naturally occurring amino acid.

In another embodiment of the present invention there is provided a compound according to formula I wherein R¹ is Ph, optionally substituted with lower alkyl, lower alkoxy, hydroxy, halo, or halo lower alkyl; R⁵ and R⁷ are hydrogen; R^{2a} is H and R^{2b} is selected from the group consisting of C₁₋₅ unbranched or branched alkyl or CH₂Ph; and R⁸ is H or lower alkyl.

In another embodiment of the present invention there is provided a compound according to formula I wherein R¹ is Np, optionally substituted with lower alkyl, lower alkoxy, hydroxy, halo, or halo lower alkyl; R⁵ and R⁷ are C(═O)R⁸ and each R⁸ is independently selected from the group consisting of C₁₋₇ unbranched or branched lower alkyl, phenyl and CH₂OPh.

In another embodiment of the present invention there is provided a compound according to formula I wherein R¹ is Ph, optionally substituted with lower alkyl, lower alkoxy, hydroxy, halo, or halo lower alkyl; R⁵ and R⁷ are COR⁸, C(═O)OR⁸, C(═O)SR⁸ or COCH(R⁸)NHR⁸; R^{2a}, R^{2b}, and R⁴ are hydrogen; R⁸ is selected from H or a group consisting of C₁₋₇ unbranched or branched lower alkyl, C₃₋₈ cycloalkyl, phenyl and the side chain of a naturally occurring amino acid.

In another embodiment of the present invention there is provided a compound according to formula I wherein R¹ is Np, optionally substituted with lower alkyl, lower alkoxy, hydroxy, halo, or halo lower alkyl; R⁵ and R⁷ are hydrogen; R^{2a} is H and R^{2b} is selected from the group consisting of C₁₋₅ unbranched or branched alkyl or CH₂Ph.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is selected from the group consisting of C₁₋₅ unbranched or branched alkyl or CH₂Ph; and R⁵ and R⁷ are both H or C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Ph, optionally substituted with lower alkyl, lower alkoxy, hydroxy, halo, or halo lower alkyl; R^{2a} is H and R^{2b} is selected from the group consisting of C₁₋₅ unbranched or branched alkyl or CH₂Ph; and R⁵ and R⁷ are both H or C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Np, optionally substituted with lower alkyl, lower alkoxy, hydroxy, halo, or halo lower alkyl; R^{2a} is H and R^{2b} is selected from the group consisting of C₁₋₅ unbranched or branched alkyl or CH₂Ph; and R⁵ and R⁷ are both H or C(═O)OR⁸; and R⁸ is H or lower alkyl.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is selected from the group consisting of C₁₋₅ unbranched or branched alkyl or CH₂Ph; and R⁵ and R⁷ are both H.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is selected from the group consisting of C₁₋₅ unbranched or branched alkyl or CH₂Ph; and R⁵ and R⁷ are both C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is methyl; and R⁵ and R⁷ are both C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is CH₂Ph; and R⁵ and R⁷ are both C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Ph or Np, optionally substituted with lower alkyl, lower alkoxy, hydroxy, halo, or halo lower alkyl; R^{2a} is H and R^{2b} is isopropyl; and R⁵ and R⁷ are both C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Np; R^{2a} is H and R^{2b} is CH₂CH(CH₃)₂; and R⁵ and R⁷ are both C(═O)R⁸; and R⁸ is lower alkyl.

In another embodiment of the present invention there is provided a method for treating diseases mediated by ZIKV comprising administering to a mammal in need thereof a therapeutically effective quantity of a compound according to formula I wherein R¹ is Ph; R^{2a} is H and R^{2b} is CH₂CH(CH₃)₂; and R⁵ and R⁷ are both C(═O)R⁸; and R⁸ is lower alkyl.

### General Phosphoramidate Schemes

WO2011092158, EP2528930, and U.S. Pat. No. 8,236,779 disclose the preparation of antiviral nucleoside phosphoramidates of 4AU for the treatment of HCV and are herein incorporated by reference in their entireties.

Phosphoramidate compounds of the present invention can be prepared by condensation of a 4′-azidouridine, compound 4, with a suitably substituted phosphochloridate, compound 3, in the presence of a strong base (Scheme 1). The nucleosides of the present invention typically contain an optionally substituted uracil and one or both of R⁵ and R⁷ are hydrogen or acyl or carbamoyl or alkoxycarbonyl. The condensation can be carried out on the unprotected nucleoside or, alternatively, the 2′,3′-hydroxy groups of the nucleoside can be protected as an acetonide or other diol protecting group known in the art. Deprotection of a nucleoside after the condensation is carried out utilizing standard protocols for nucleic acid chemistry.

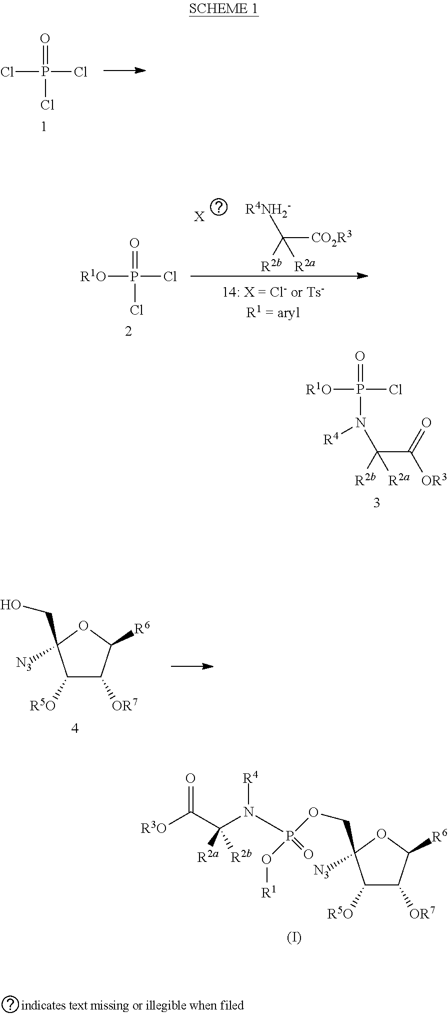

The requisite substituted phosphochloridate compounds 3 utilized to prepare compounds of the present invention are prepared by a two-step sequence comprising condensation of phosphorus oxychloride (1) with a suitably substituted phenol to afford an aryloxy phosphorodichloridates 2 which are subsequently treated with acid addition salt of an α-amino acid ester in the presence of TEA to afford an aryloxy phosphorochloridate 3 (for representative procedure see, e.g., D. Curley et al. Antiviral Res. 1990 14:345-356; C. McGuigan et al. Antiviral Res. 1992 17:311-321; McGuigan et al. Antiviral Chem. Chemother 1990 1(2):107-113).

Condensation of aryloxy phosphorochloridate 3 with a nucleoside 4 wherein R⁶ is optionally substituted uridine, and one or both of R⁵ and R⁷ are hydrogen or acyl or carbamoyl or alkoxycarbonyl. When R⁵ and R⁷ are both hydrogen, 2′,3′-diol can form an acetal or ketal protecting group. Treating a nucleoside with an aryloxy phosphoramidate in the presence of strong base affords the phosphoramidate derivatives of the invention (for representative procedures see, e.g. K. S. Gudmundsson, Nucleosides, Nucleotides & Nucleic Acids 2003 22(10):1953-1961). When 2′,3′-diol are protected by an acetal or ketal group, a subsequent deprotection step is required which steps are know in the art.

Compounds of formula I may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

The term “amino acid” as used herein refers to naturally occurring α amino carboxylic acids, as well as to optical isomers (enantiomers and diastereomers), synthetic analogs and derivatives thereof. α-Amino acids comprise a carbon atom bonded to a carboxyl group, an amino group, a hydrogen atom and a unique “side chain” group. The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. The side chains of naturally occurring amino acids include: hydrogen, methyl, iso-propyl, iso-butyl, sec-butyl, —CH₂OH, —CH(OH)CH₃, —CH₂SH, —CH₂CH₂SMe, —(CH2)pCOR wherein R is —OH or —NH₂ and p is 1 or 2, —(CH2)q-NH₂ where q is 3 or 4, —(CH₂)₃—NHC(═NH)NH₂, —CH₂C₆H₅, —CH₂-p-C₆H₄—OH, (3-indolinyl)methylene, (4-imidazolyl)methylene.

Compounds of the present invention may have asymmetric centers located on the side chain of a carboxylic ester, amide or carbonate moiety that produce diastereomers when linked to the nucleoside. All stereoisomers of a side chain of compounds of the instant invention are contemplated, either in admixture or in pure or substantially pure form. The definition of the compounds according to the invention embraces all both isolated optical isomers enantiomers and their mixtures including the racemic form. The pure optical isomer can be prepared by stereospecific synthesis from α-D-ribose or the racemic form can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

### PREPARATIVE EXAMPLES

### Example 1 (Compounds I-1 and I-3)

The process chemistry route for the 4AU parent nucleoside has been established and is used as starting material for the synthesis of I-1 and I-3, following Scheme 2.

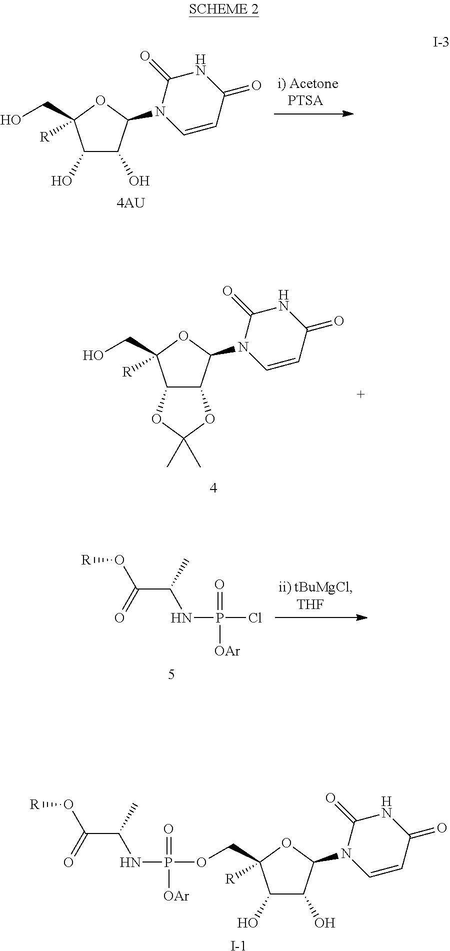

### Example 2 (Compound I-34)

### Ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate

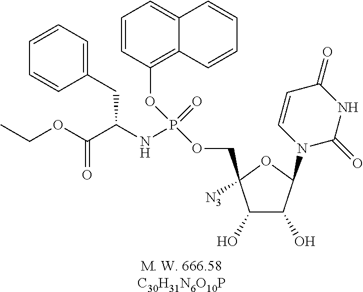 [SMILES: CCOC(=O)[C@H](Cc1ccccc1)NP(=O)(OC[C@@]1(N=[N+]=[N-])O[C@@H](n2ccc(=O)[nH]c2=O)[C@H](O)[C@@H]1O)Oc1cccc2ccccc12]

The titled compound was prepared in a similar manner to the methods described by McGuigan, Christopher et al in Journal of Medicinal Chemistry (2007), 50(8), 1840-1849.

### Example 3 (Compound I-35)

### Benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate

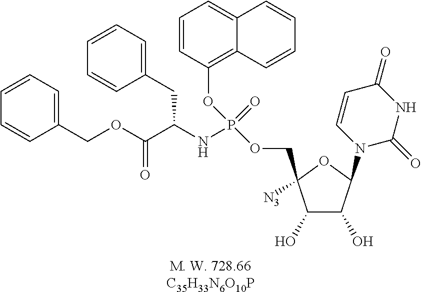 [SMILES: [N-]=[N+]=N[C@]1(COP(=O)(N[C@@H](Cc2ccccc2)C(=O)OCc2ccccc2)Oc2cccc3ccccc23)O[C@@H](n2ccc(=O)[nH]c2=O)[C@H](O)[C@@H]1O]

The titled compound was prepared in a similar manner to the methods described by McGuigan, Christopher et al in Journal of Medicinal Chemistry (2007), 50(8), 1840-1849.

### BIOLOGICAL EXAMPLES

### Antiviral Activity

Another aspect of the invention relates to methods of inhibiting viral infections, comprising the step of treating a sample or subject suspected of needing such inhibition with a composition of the invention.

Within the context of the invention samples suspected of containing a virus include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing an organism which induces a viral infection, frequently a pathogenic organism such as a tumor virus. Samples can be contained in any medium including water and organic solvent\water mixtures. Samples include living organisms such as humans, and man-made materials such as cell cultures.

If desired, the anti-virus activity of a compound of the invention after application of the composition can be observed by any method including direct and indirect methods of detecting such activity. Quantitative, qualitative, and semi-quantitative methods of determining such activity are all contemplated. Typically one of the screening methods described above are applied, however, any other method such as observation of the physiological properties of a living organism are also applicable.

The antiviral activity of a compound of the invention can be measured using standard screening protocols that are known. For example, the antiviral activity of a compound can be measured using the following general protocols.

### Renilla Luciferase (Rluc) Assay

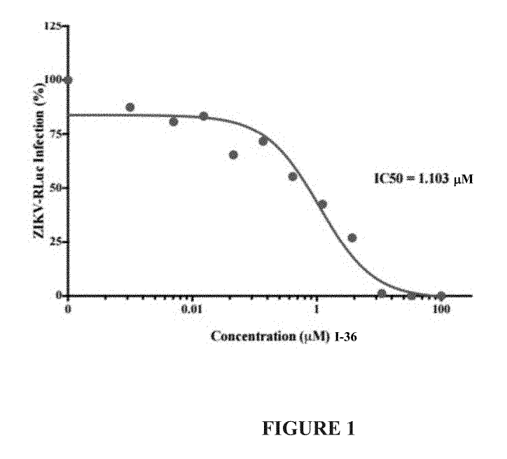

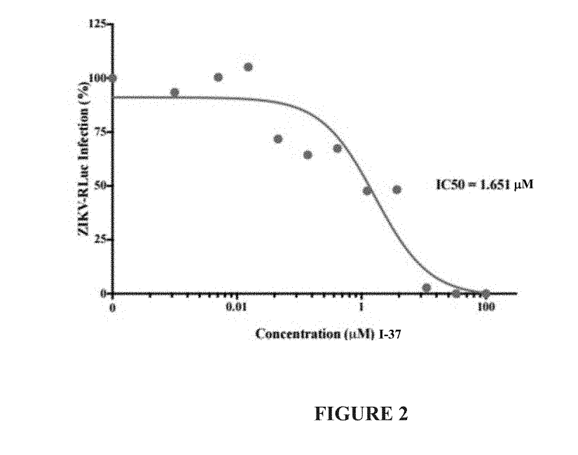

Single Epimers I-36 and I-37, of I-1 were tested in the Rluc assay at 72 h.p.i. for % inhibition vs. concentration of compound (FIGS. 1-2):

| Compound ID | IC50 Zika (μM) |
|---|---|
| I-36 | 1.103 |
| I-37 | 1.651 |

### Standard Huh7 Cells Antiviral Assay for Zika Virus

The human hepatoma cell line Huh-7, are cultured in DMEM without phenol-red (Cellgro Mediatech, Cat #10-013-CV containing 4.5 g/l glucose, L-glutamine & sodium pyruvate). The medium is further supplemented with 10% (v/v) FBS (ATLAS Cat # F-0500-A, lot#850114A) and 1% (vlv) penicillin/streptomycin (Cellgro Mediatech #30-022-CI). Cells are maintained at 37° C. in a humidified 5% CO₂ atmosphere. Zika virus representative strain titers are measured on BHK-21 cells, using a standard plaque assay procedure. For the determination of EC50 of nucleoside in the antiviral assay, Huh-7 cells are plated in white 96-well plates in MEM media supplemented with 10% FBS and 1% penicillin/streptomycin. After incubation for 24 h, the cells are infected at a multiplicity of infection (MOI) of 0.5 for 2 h at 37° C. Ten three-fold dilutions of compounds are prepared in the same media supplemented with 1% DMSO. After the 2 h adsorption phase, the virus is aspirated off and diluted compound is added to four wells each. Huh-7 cells are plated as described above and exposed to the same concentration range of compounds. Untreated cells are carried along as a control. After a 3-day incubation at 37° C., the cell viability is determined using Cell-titer Glo™ reagent (Promega, Madison, Wis.) that is added to each well and incubated for 5 min. Plates are analyzed using a Thermo Luminoskan plate reader (Waltham, Mass.).

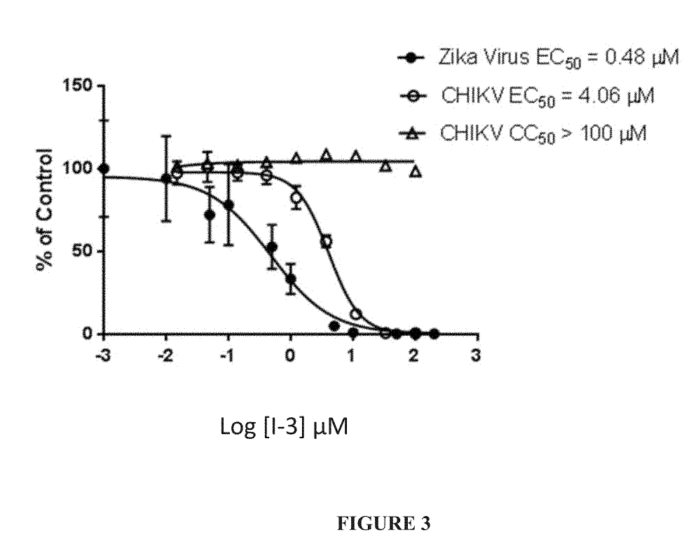

Antiviral activity of I-3 (prodrug of 4AU) against ZIKV and CHIKV replication and cell viability determined in Huh-7 cell derived CHIKV replicon cells by intracellular ATP quantification (CellTiter-Glo). Mean values shown as individual data points; standard deviations as error bars (FIG. 3).

Prodrugs are passaged in Huh-7 cells at increasing drug concentrations to select ZIKV variants resistant to the prodrug and to determine polymerase mutations that could confer resistance to the drug. Recombinant ZIKV polymerase can be expressed and purified in E. coli and the mechanism of action of 4AUTP as an inhibitor of the viral polymerase may be demonstrated; if potential resistance mutations are identified from passaging, they may be validated by demonstrating resistance using recombinant ZIKV polymerases with site directed mutations. The prodrug of the instant application tested for activity against ZIKV demonstrated antiviral activity at non-cytotoxic concentrations with EC50<5 μM in human cell types that are relevant to ZIKV pathogenicity in humans (e.g. neuronal, placental cells). Virus passaging experiments with at least 12 passaging steps were completed and level of resistance and polymerase sequences from emerging virus variants were determined. Enzymatically active recombinant ZIKV polymerase were generated and 4AUTP Ki values and Ki/Km ratios were determined.

Methods: Cytotoxicity Assays:

Primary human PBMC are obtained from at least 3 donors (Stemcell Technologies) and cultured in the presence of phytohaemagglutinin and IL2 to induce cell proliferation. The cells are incubated with test compounds for 6 days, after which the cell number (counting) and cell viability (ATP quantitation, CellTiter Glo, Promega) are determined. AZT (Zidovudine) and Ribavirin are used as controls of cytotoxic compounds. The proliferation of human CD34+ bone marrow stem cells is performed according to the protocol provided by AllCells, Alameda, Calif. The inhibition of bone marrow stem cell proliferation is determined after 6 days of incubation with test compound using AZT and Ribavirin as positive control inhibitors. Human cardiomyocytes are obtained from Cellular Dynamics International and are cultured in vitro for 6 days in the presence of test compounds. Cell viability is determined by ATP quantitation (CellTiterGlo, Promega) at the end of treatment. AZT and Ribavirin are used as positive control inhibitors. Permeability in Caco-2 cells assay is performed using standard methods.

Determination of Intracellular Triphosphate Concentrations:

The formation of 4AUTP triphosphate is determined in human cells (Huh-7 cells, PBMC), and representative mouse, rat, dog and cynomolgus monkey cells. The intracellular triphosphate concentration is determined using LC-MS/MS. In human cells, the time and concentration dependent formation of 4AUTP triphosphate is determined, as well as the half-life after removal of all extracellular prodrug by exchange of cell culture medium. Analysis of intracellular nucleoside phosphates is conducted using well-established methods.

Prodrugs were tested to determine selectivity against human polymerases and potential for mitochondrial toxicity in human cells. I-1 and I-3 did not inhibit the activity of human RNA polymerases I, II or III in human HeLa cells determined using RNA polymerase I, II, and III specific reporter gene assays (no inhibition at 10 or 100 μM compound concentrations that represented highest soluble concentrations in cell culture medium). No mitochondrial toxicity was indicated after prolonged (up to 11-day) incubation of human HepG2 cells with 4AU or 4AU prodrugs.

TABLE 2
Antiviral activity against ZIKV strain MR766 (Genbank LC002520)
replication determined in human Huh-7 or mouse MEF cells, and against HCV replicons of
genotype 1a (H77) and 1b (Con1) and Chikungunya replicon (CHIKV strain LR2006 OPY-1,
GenBank EU224270). (FIG. 3)

| | ZIKV | | HCV GT1a | | HCV GT1b | | CHIKV | |
|---|---|---|---|---|---|---|---|---|
| | EC50 (μM) | CC50 (μM) | EC50 (μM) | CC50 (μM) | EC50 (μM) | CC50 (μM) | EC50 (μM) | CC50 (μM) |
| I-1 | 0.74 | >10* | 0.42 | >10* | 0.33 | >10* | nt | nt |
| I-3 | 0.48 | >100 | nt | nt | 0.40 | >100 | 4.1 | >100 |
| 4AU | nt | nt | nt | nt | 217 | >1000 | >100 | >1000 |

*I-1 was not soluble in cell culture medium at concentrations above 10 μM. I-3 is a 4AU prodrug with higher intrinsic solubility in aqueous media. 4AU has higher aqueous solubility as compared to its monophosphate prodrugs.

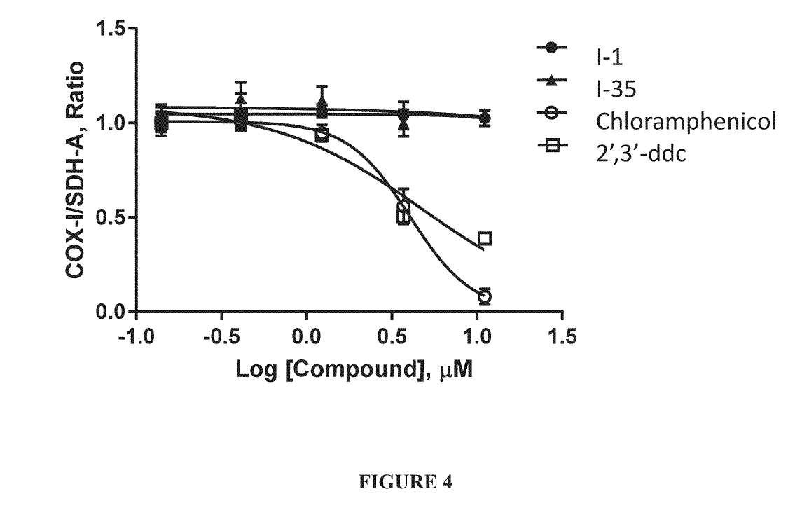

Mitochondrial toxicity was determined in HepG2 cells after 11 days of incubation with nucleoside analogs. Mitochondrial toxicity is apparent as a reduction in mitochondrially encoded protein COX-I as compared to nuclear encoded protein SDH-A. A decrease of the ratio between these two proteins indicates mitochondrial toxicity as shown by reference compounds chloramphenicol and 2′,3′-dideoxy-cytidine. Mean values shown as individual data points; standard deviations as error bars (FIG. 4). Nucleoside-associated toxicity has been ascribed mainly to the inhibition of human polymerases by the triphosphate (TP) derivatives of nucleoside analogs. 4AUTP, the active viral polymerase inhibitor delivered by the 4AU prodrugs, did not appreciably inhibit the mitochondrial DNA polymerase pol γ (IC50>1 mM). In comparison, the antiviral nucleosides approved for the treatment of HIV infection, AZT triphosphate and lamivudine triphosphate fully inhibited DNA pol γ with mean IC50 values of 2.47 μM and 149 μM, respectively. 4AUTP also did not inhibit human DNA polymerase α or β (IC50>1 mM) and human RNA polymerase I, II, or III activities in cell extracts (IC50>1 mM), consistent with the cell based reporter gene assay results. Lack of inhibition of human RNA polymerases by ribonucleoside analogs I-1 and I-3, and lack of mitochondrial toxicity in human cells predicts a good nucleoside analog safety profile in humans.

In order to evaluate genotoxicity of the lead nucleoside analog, a complete genotoxicity assessment had been performed by Roche on the nucleoside analog 4AU, including in vitro AMES test, CHO/HGPRT mammalian gene mutation test, chromosomal aberration test, and in vivo micronucleus and comet assays in rats at doses up to 2000 mg/kg/day. The weight of evidence from these studies indicated that 4AU is not genotoxic.

The tested prodrugs of Formula I have shown similar antiviral activity against ZIKV and HCV, both from the Flaviviridae family. Nucleoside analogs targeting HCV have demonstrated antiviral activity across all HCV genotypes and existing HCV strains and have also shown a high barrier to resistance in cell culture and in clinical studies. It is therefore expected that such prodrugs will be broadly active across existing and future ZIKV strains and will have a high barrier to resistance development. To generate data to support this expected profile: a) prodrugs are tested against available ZIKV strains in cell culture and then are tested against ZIKV in primary human cells including PBMC and dendritic cells and in neuronal progenitor and placental cells, and b) in vivo in the mouse model of lethal ZIKV infection (developed by Nagamine). Viral replication in and survival of ZIKV challenged mice treated with increasing doses of 4AU prodrug, with treatment starting at different times after infection, is measured. A mouse model in which mice are infected with ZIKV during pregnancy allows for testing the effect of nucleoside treatment in preventing congenital malformations.

Permeability in Caco2 cells has been associated with increased likelihood of achieving high plasma concentrations of nucleoside analogs after oral dosing. Nucleoside toxicity in clinical studies has been associated with lack of selectivity against human polymerases, cytotoxicity in primary human cells and mitochondrial toxicity. The 4AU nucleoside structure contains known features to confer selectivity against human polymerases, and that selectivity has been demonstrated in assays using purified and recombinant human DNA polymerases alpha, beta and gamma, and human RNA polymerases in human cell extracts. The selectivity of the 4AU prodrug has in addition been demonstrated in cell-based human RNA polymerase I, II, and III-dependent reporter gene assays and mitochondrial toxicity has been determined with both 4AU and prodrugs in HepG2 cell incubations up to 11 days duration.

### Summary of Results

I-1 and I-3 are two prodrugs with different physicochemical properties that deliver the same nucleoside (4′-azido-uridine triphosphate, 4AU) to multiple human target cells. 4AU itself is not efficiently converted to 4AU-triphosphate (4AUTP) in human cells. Therefore, I-1 and I-3 were developed as monophosphate prodrugs of 4AU that can deliver the nucleoside analog in a fashion capable of being converted to its active triphosphate form across multiple human cell types resulting in antiviral activity across the different target cells that ZIKV can infect. 4AU was originally discovered and characterized by Hoffmann La-Roche as a nucleoside inhibitor of different positive strand RNA viruses. 4AUTP has shown inhibition of HCV and Dengue virus polymerase, but is not active against influenza and RSV polymerases. The safety of 4AU has already been demonstrated in chronic (13 week) preclinical GLP toxicity studies in rats and monkeys and in a Phase 1 clinical study in humans at doses that showed significant antiviral activity against HCV replication. 4AU has also demonstrated lack of mitochondrial toxicity in human cells, consistent with the preclinical and human safety profile of 4AU. The application of prodrug chemistry to 4AU, has enabled the generation of a drug candidate for the treatment and prophylaxis of ZIKV infection that can meet the above described target profile of an effective treatment for this indication.

In conclusion, the prodrugs disclosed herein, for example 1) I-36 and I-37, single epimers of I-1, exhibited low micromolar IC50 values, 1.103 μM and 1.651 μM, respectively, in ZIKV-RLuc % inhibition per concentration prodrug over 72 h.p.i. (FIGS. 1-2); 2) I-1 and I-3, exhibited high antiviral inhibition potency (submicromolar EC50), with no cytotoxicity in cell culture (Huh-7 cell CC50>100 uM); and thus, 3) the monophosphate prodrugs of 4AU disclosed in the instant application have, as described herein, exhibited potency against ZIKV in both in vitro and cellular assays; wherein 4) the preclinical and clinical safety of 4AU has already been tested and determined; and thus 5) the 4AU prodrugs described herein combine the features of a potent and direct antiviral profile that offers the opportunity to provide safe and effective treatment and prophylaxis of ZIKV infection (see Table 2).

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application, in particular WO2002100415, WO2004046159, WO2007020193, WO2011092158, WO2012168348, WO2013092447, and WO2014193663 are hereby incorporated by reference in their entirety for all purposes to the same extent as if each individual patent, patent application or publication were so individually denoted.

We claim:

1. A method of preventing or treating Zika Virus comprising administering to a patient in need thereof a compound selected from the group consisting of:
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
tert-butyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
(2R,3S,4R,5R)-2-azido-2-((((((S)-1-(benzyloxy)-1-oxopropan-2-yl)amino)(naphthalen-1-yloxy)phosphoryl)oxy)methyl)-5-(2,4-dioxo-3,4-dihydropyrimidin-1(2H)-yl)tetrahydrofuran-3,4-diyl dipropionate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((4-methoxynaphthalen-1-yl)oxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-leucinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-leucinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((1-bromonaphthalen-2-yl)oxy)phosphoryl)-L-leucinate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)glycinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)((3-methoxynaphthalen-2-yl)oxy)phosphoryl)-L-alaninate;
ethyl N-((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-O-methyl-L-homoserinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-valinate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(4-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(2-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(3-(trifluoromethyl)phenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(4-cyanophenoxy)phosphoryl)-L-alaninate;
isopropyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl 1-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)cyclopentane-1-carboxylate;
isopropyl 1-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)cyclopentane-1-carboxylate;
ethyl 2-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)-2-methylpropanoate;
isopropyl 2-(((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)amino)-2-methylpropanoate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(3-cyanophenoxy)phosphoryl)-L-alaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(2-cyanophenoxy)phosphoryl)-L-alaninate;
(2R,3S,4R,5R)-2-azido-2-((((((S)-1-(tert-butoxy)-1-oxopropan-2-yl)amino)(naphthalen-1-yloxy)phosphoryl)oxy)methyl)-5-(2,4-dioxo-3,4-dihydropyrimidin-1(2H)-yl)tetrahydrofuran-3,4-diyl dipropionate;
(2R,3S,4R,5R)-2-azido-2-((((((S)-1-(tert-butoxy)-1-oxopropan-2-yl)amino)(phenoxy)phosphoryl)oxy)methyl)-5-(2,4-dioxo-3,4-dihydropyrimidin-1(2H)-yl)tetrahydrofuran-3,4-diyl dipropionate;
2,2,2-trifluoroethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
2,2,2-trifluoroethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(phenoxy)phosphoryl)-L-alaninate;
benzyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
tert-butyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
tert-butyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(naphthalen-1-yloxy)phosphoryl)-L-alaninate;
isopropyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
isopropyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(naphthalen-1-yloxy)phosphoryl)-L-alaninate;
ethyl ((((2R,3S,4R,5R)-2-azido-3,4-dihydroxy-5-(5-methyl-2,4-dioxo-3,4-dihydropyrimidin-1(2H)-yl)tetrahydrofuran-2-yl)methoxy)(phenoxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate;
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-phenylalaninate;
benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate; and
((((2R,3S,4R,5R)-2-azido-5-(2,4-dioxo-3,4-dihydropyrimidin-1(2H)-yl)-3,4-dihydroxytetrahydrofuran-2-yl)methoxy)(hydroxy)phosphoryl)-L-alanine;
or a pharmaceutically acceptable salt thereof.

2. A method for preventing or treating Zika Virus comprising administering to a patient in need thereof a compound of Formula I 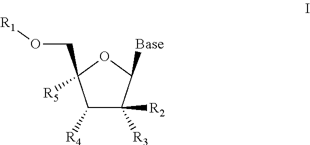
wherein:
R₁ is hydrogen, ethyl ester, or monophrate, diphosphate, triphosphate, or phosphoramidate;
R2 is F, Me, or H;
R3 is OH, F, or H;
R4 is OH, F, or H;
R5 is azido, difluoromethyl, fluoromethyl, vinyl, ethyl, or H; and
Base is uracil, optionally substituted with methyl or Br;
or pharmaceutically acceptable salts thereof.

3. The method of claim 1, further comprising administering at least one other antiviral agent.

4. The method of claim 2, further comprising administering at least one other antiviral agent.

5. A method of preventing or treating Zika Virus by administering to a patient in need thereof a composition comprising the compound of claim 1.

6. A method of preventing or treating Zika Virus by administering to a patient in need thereof a composition comprising the compound of claim 2.

7. A method of preventing or treating Zika Virus by administering to a patient in need thereof a composition comprising the compound of claim 1, admixed with a diluent.

8. A method of preventing or treating Zika Virus by administering to a patient in need thereof a composition comprising the compound of claim 2, admixed with a diluent.

9. A method of preventing or treating Zika Virus by administering to a patient in need thereof a composition comprising the compound of claim 1, further comprising administering at least one other antiviral agent.

10. A method of preventing or treating Zika Virus by administering to a patient in need thereof a composition comprising the compound of claim 2, further comprising administering at least one other antiviral agent.

11. A method of preventing or treating Zika Virus by administering to a patient in need thereof a compound selected from the group consisting of:
benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate; and
benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate;
or a pharmaceutically acceptable salt thereof.

12. The method of claim 11, wherein the compound is benzyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

13. The method of claim 11, wherein the compound is ethyl ((((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

14. The method of claim 11, wherein the compound is benzyl ((R)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.

15. The method of claim 11, wherein the compound is benzyl ((S)-(((2R,3S,4R,5R)-2-azido-5-(2,4-dioxo-3,4-dihydropyrimidin-1(2H)-yl)-3,4-dihydroxytetrahydrofuran-2-yl)methoxy)(naphthalen-1-yloxy)phosphoryl)-L-alaninate or a pharmaceutically acceptable salt thereof.
